(19) United States
(12) Patent Application Publication
(10) Pub. No.: US 2006/0193912 A1
(43) Pub. Date: Aug. 31, 2006

(54) Controlled release O-desmethylvenlafaxine formulations

(75) Inventors: Sara Ketsela, Danbury, CT (US); Dean Dinicola, Warren, CT (US); Anand R. Baichwal, Wappingers Falls, NY (US)

Correspondence Address: DAVIDSON, DAVIDSON & KAPPEL, LLC, 485 SEVENTH AVENUE, 14TH FLOOR, NEW YORK, NY, 10018, US

(73) Assignee: Penwest Pharmaceuticals Co., Patterson, NY (US)

(21) Appl. No.: 11/363,842

(22) Filed: Feb. 28, 2006

Related U.S. Application Data
(60) Provisional application No. 60/657,035, filed on Feb. 28, 2005.
(60) Provisional application No. 60/750,594, filed on Dec. 14, 2005.

(51) Int. Cl. A61K 9/22

(52) U.S. Cl. 424/468

(57) ABSTRACT

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 1 hour in 900 mL 0.1 N HCl (pH 1.5) with 40% EtOH using USP Apparatus II at 50 rpm is within 25% of the amount of O-desmethylvenlafaxine or pharmaceutically acceptable salt thereof released at 1 hour in 900 mL 0.1 N HCl (pH 1.5) using USP Apparatus II at 50 rpm.

This application claims priority from U.S. Provisional Application Nos. 60/657,035, filed on Feb. 28, 2005; and 60/750,594, filed on Dec. 14, 2005; the disclosures of which are incorporated by reference in their entireties.

### FIELD OF THE INVENTION

The present invention relates to controlled release dosage forms containing a therapeutically effective amount of O-desmethylvenlafaxine hydrochloride which are resistant to alcohol induced dose dumping.

### BACKGROUND OF THE INVENTION

The advantages of controlled release products are well known in the pharmaceutical field and include the ability to maintain a desired blood level of a medicament over a comparatively longer period of time while increasing patient compliance by reducing the number of administrations necessary to achieve the same. These advantages have been attained by a wide variety of methods.

For example, different hydrogels have been described for use in controlled release medicines, some of which are synthetic, but most of which are semi-synthetic or of natural origin. A few contain both synthetic and non-synthetic material. However, some of the systems require special process and production equipment, and in addition some of these systems are susceptible to variable drug release.

Oral controlled release delivery systems should ideally be adaptable so that release rates and profiles can be matched to physiological and chronotherapeutic requirements.

While many controlled and sustained-release formulations are already known, certain soluble to highly soluble drugs present formulation difficulties when included in such formulation. An example of such a highly soluble drug is venlafaxine hydrochloride.

Currently, venlafaxine is available as 150 mg, 75 mg, and 37.5 mg extended release capsules and is marketed under the name Effexor XR® by Wyeth-Ayerst Company.

Effexor XR® is susceptible to alcohol induced dose dumping. Accordingly, if the dosage form is administered with an amount of alcohol, the integrity of the controlled release mechanism of the dosage form will be compromised and a potentially toxic amount of venlafaxine may be available for immediate release.

Accordingly, there exists a need in the art to provide a controlled release oral tablet that provides for the extended release of venlafaxine hydrochloride which has reduced potential for alcohol induced dose dumping. All documents cited herein, including the foregoing, are incorporated by reference in their entireties for all purposes.

### OBJECTS AND SUMMARY OF THE INVENTION

It is an object of the present invention to provide an oral controlled release release formulation comprising O-desmethylvenlafaxine or a pharmaceutically acceptable salt thereof which has reduced potential for alcohol induced dose dumping.

It is an object of certain embodiments of the present invention to provide an oral controlled release formulation comprising O-desmethylvenlafaxine or a pharmaceutically acceptable salt thereof which has reduced potential for alcohol induced dose dumping and is suitable for once-a-day administration.

It is a further object of certain embodiments of the present invention to provide an oral controlled release dosage form which has reduced potential for alcohol induced dose dumping and which releases O-desmethylvenlafaxine or pharmaceutically acceptable salt thereof over an extended period of time, e.g., for a time period of at least about 24 hours, when the tablets are exposed to an environmental fluid (e.g., the gastrointestinal tract).

It is a further object of certain embodiments of the present invention to provide a controlled release oral dosage form which has reduced potential for alcohol induced dose dumping as compared to a commercialized extended release dosage form comprising venlafaxine hydrochloride (e.g., Effexor XR).

It is a further object of certain embodiments of the present invention to provide a controlled release oral dosage form which has reduced potential for alcohol induced dose dumping as compared to the extended release dosage form comprising venlafaxine hydrochloride approved by the FDA under NDA application No. 020699 on Oct. 20, 1997.

It is a further object of certain embodiments of the present invention to provide methods for preparing the O-desmethylvenlafaxine controlled release oral dosage forms disclosed herein.

It is a further object of certain embodiments of the present invention to provide a method of treating depression comprising administering the O-desmethylvenlafaxine controlled release oral dosage forms disclosed herein.

It is a further object of certain embodiments of the present invention to provide a method for reducing the level of nausea and/or incidence of emesis associated with multiple daily dosing of venlafaxine hydrochloride, which comprises dosing a patient in need of treatment with O-desmethylvenlafaxine, the controlled release oral dosage forms disclosed herein.

It is a further object of certain embodiments of the present invention to provide a method of reducing alcohol induced dose dumping of O-desmethylvenlafaxine from a controlled release dosage form comprising preparing a controlled release dosage form disclosed herein.

The above-mentioned objects and others are achieved by virtue of the present invention, which is directed in part to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 1 hour in 900 mL 0.1 N HCl (pH 1.5) with 40% EtOH using USP Apparatus II at 50 rpm is within 25%, within 15%, within 10%, or within 5% of the amount of O-desmethylvenlafaxine or pharmaceutically acceptable salt thereof released at 1 hour in 900 mL 0.1 N HCl (pH 1.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 2 hours in 900 mL 0.1 N HCl (pH 1.5) with 40% EtOH using USP Apparatus II at 50 rpm is within 25%, within 15%, within 10%, or within 5% of the amount of O-desmethylvenlafaxine or pharmaceutically acceptable salt thereof released at 2 hours in 900 mL 0.1 N HCl (pH 1.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 4 hours in 900 mL 0.1 N HCl (pH 1.5) with 40% EtOH using USP Apparatus II at 50 rpm is within 25%, within 15%, within 10%, or within 5% of the amount of O-desmethylvenlafaxine or pharmaceutically acceptable salt thereof released at 4 hours in 900 mL 0.1 N HCl (pH 1.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 8 hours in 900 mL 0.1 N HCl (pH 1.5) with 40% EtOH using USP Apparatus II at 50 rpm is within 25%, within 15%, within 10%, or within 5% of the amount of O-desmethylvenlafaxine or pharmaceutically acceptable salt thereof released at 8 hours in 900 mL 0.1 N HCl (pH 1.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 16 hours in 900 mL 0.1 N HCl (pH 1.5) with 40% EtOH using USP Apparatus II at 50 rpm is within 25%, within 15%, within 10%, or within 5% of the amount of O-desmethylvenlafaxine or pharmaceutically acceptable salt thereof released at 16 hours in 900 mL 0.1 N HCl (pH 1.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 24 hours in 900 mL 0.1 N HCl (pH 1.5) with 40% EtOH using USP Apparatus II at 50 rpm is within 25%, within 15%, within 10%, or within 5% of the amount of O-desmethylvenlafaxine or pharmaceutically acceptable salt thereof released at 24 hours in 900 mL 0.1 N HCl (pH 1.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 1 hour in 900 mL 0.1 N HCl (pH 1.5) with 40% EtOH using USP Apparatus II at 50 rpm to the amount of O-desmethylvenlafaxine or pharmaceutically acceptable salt thereof released at 1 hour in 900 mL 0.1 N HCl (pH 1.5) using USP Apparatus II at 50 rpm is no more than 1.5:1; no more than about 1.2:1.

In certain embodiments, the present invention is directed to a controlled release oral dosage form comprising: a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 2 hours in 900 mL 0.1 N HCl (pH 1.5) with 40% EtOH using USP Apparatus II at 50 rpm to the amount of O-desmethylvenlafaxine or pharmaceutically acceptable salt thereof released at 2 hours in 900 mL 0.1 N HCl (pH 1.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 4 hours in 900 mL 0.1 N HCl (pH 1.5) with 40% EtOH using USP Apparatus II at 50 rpm to the amount of O-desmethylvenlafaxine or pharmaceutically acceptable salt thereof released at 4 hours in 900 mL 0.1 N HCl (pH 1.5) using USP Apparatus II at 50 rpm is no more than 1.5:1; no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 8 hours in 900 mL 0.1 N HCl (pH 1.5) with 40% EtOH using USP Apparatus II at 50 rpm to the amount of O-desmethylvenlafaxine or pharmaceutically acceptable salt thereof released at 8 hours in 900 mL 0.1 N HCl (pH 1.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 16 hours in 900 mL 0.1 N HCl (pH 1.5) with 40% EtOH using USP Apparatus II at 50 rpm to the amount of O-desmethylvenlafaxine or pharmaceutically acceptable salt thereof released at 16 hours in 900 mL 0.1 N HCl (pH 1.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 24 hours in 900 mL 0.1 N HCl (pH 1.5) with 40% EtOH using USP Apparatus II at 50 rpm to the amount of O-desmethylvenlafaxine or pharmaceutically acceptable salt thereof released at 24 hours in 900 mL 0.1 N HCl (pH 1.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 1 hour in 900 mL 0.1 N HCl (pH 1.5) with 40% EtOH using USP Apparatus II at 50 rpm is less than the amount of O-desmethylvenlafaxine or pharmaceutically acceptable salt thereof released at 1 hour in 900 mL 0.1 N HCl (pH 1.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 2 hours in 900 mL 0.1 N HCl (pH 1.5) with 40% EtOH using USP Apparatus II at 50 rpm is less than the amount of O-desmethylvenlafaxine or pharmaceutically acceptable salt thereof released at 2 hours in 900 mL 0.1 N HCl (pH 1.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 4 hours in 900 mL 0.1 N HCl (pH 1.5) with 40% EtOH using USP Apparatus II at 50 rpm is less than the amount of O-desmethylvenlafaxine or pharmaceutically acceptable salt thereof released at 4 hours in 900 mL 0.1 N HCl (pH 1.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 8 hours in 900 mL 0.1 N HCl (pH 1.5) with 40% EtOH using USP Apparatus II at 50 rpm is less than the amount of O-desmethylvenlafaxine or pharmaceutically acceptable salt thereof released at 8 hours in 900 mL 0.1 N HCl (pH 1.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 16 hours in 900 mL 0.1 N HCl (pH 1.5) with 40% EtOH using USP Apparatus II at 50 rpm is less than the amount of venlafaxine or pharmaceutically acceptable salt thereof released at 16 hours in 900 mL 0.1 N HCl (pH 1.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 24 hours in 900 mL 0.1 N HCl (pH 1.5) with 40% EtOH using USP Apparatus II at 50 rpm is less than the amount of O-desmethylvenlafaxine or pharmaceutically acceptable salt thereof released at 24 hours in 900 mL 0.1 N HCl (pH 1.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 1 hour in 900 mL 0.1 N HCl (pH 4.5) with 40% EtOH using USP Apparatus II at 50 rpm is within 25%, within 15%, within 10%, or within 5% of the amount of O-desmethylvenlafaxine or pharmaceutically acceptable salt thereof released at 1 hour in 900 mL 0.1 N HCl (pH 4.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 2 hours in 900 mL 0.1 N HCl (pH 4.5) with 40% EtOH using USP Apparatus II at 50 rpm is within 25%, within 15%, within 10%, or within 5% of the amount of O-desmethylvenlafaxine or pharmaceutically acceptable salt thereof released at 2 hours in 900 mL 0.1 N HCl (pH 4.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 4 hours in 900 mL 0.1 N HCl (pH 4.5) with 40% EtOH using USP Apparatus II at 50 rpm is within 25%, within 15%, within 10%, or within 5% of the amount of O-desmethylvenlafaxine or pharmaceutically acceptable salt thereof released at 4 hours in 900 mL 0.1 N HCl (pH 4.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 8 hours in 900 mL 0.1 N HCl (pH 4.5) with 40% EtOH using USP Apparatus II at 50 rpm is within 25%, within 15%, within 10%, or within 5% of the amount of O-desmethylvenlafaxine or pharmaceutically acceptable salt thereof released at 8 hours in 900 mL 0.1 N HCl (pH 4.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 16 hours in 900 mL 0.1 N HCl (pH 4.5) with 40% EtOH using USP Apparatus II at 50 rpm is within 25%, within 15%, within 10%, or within 5% of the amount of O-desmethylvenlafaxine or pharmaceutically acceptable salt thereof released at 16 hours in 900 mL 0.1 N HCl (pH 4.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 24 hours in 900 mL 0.1 N HCl (pH 4.5) with 40% EtOH using USP Apparatus II at 50 rpm is within 25%, within 15%, within 10%, or within 5% of the amount of O-desmethylvenlafaxine or pharmaceutically acceptable salt thereof released at 24 hours in 900 mL 0.1 N HCl (pH 4.5) using USP Apparatus II at 50 rpm.

In certain embodiments, the present invention is directed to a controlled release oral dosage form comprising: a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 1 hour in 900 mL 0.1 N HCl (pH 4.5) with 40% EtOH using USP Apparatus II at 50 rpm to the amount of O-desmethylvenlafaxine or pharmaceutically acceptable salt thereof released at 1 hour in 900 mL 0.1 N HCl (pH 4.5) using USP Apparatus II at 50 rpm is no more than 1.5:1; no more than about 1.2:1.

In certain embodiments, the present invention is directed to a controlled release oral dosage form comprising: a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 2 hours in 900 mL 0.1 N HCl (pH 4.5) with 40% EtOH using USP Apparatus II at 50 rpm to the amount of venlafaxine or pharmaceutically acceptable salt thereof released at 2 hours in 900 mL 0.1 N HCl (pH 4.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 4 hours in 900 mL 0.1 N HCl (pH 4.5) with 40% EtOH using USP Apparatus II at 50 rpm to the amount of O-desmethylvenlafaxine or pharmaceutically acceptable salt thereof released at 4 hours in 900 mL 0.1 N HCl (pH 4.5) using USP Apparatus II at 50 rpm is no more than 1.5:1; no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 8 hours in 900 mL 0.1 N HCl (pH 4.5) with 40% EtOH using USP Apparatus II at 50 rpm to the amount of O-desmethylvenlafaxine or pharmaceutically acceptable salt thereof released at 8 hours in 900 mL 0.1 N HCl (pH 4.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 16 hours in 900 mL 0.1 N HCl (pH 4.5) with 40% EtOH using USP Apparatus II at 50 rpm to the amount of O-desmethylvenlafaxine or pharmaceutically acceptable salt thereof released at 16 hours in 900 mL 0.1 N HCl (pH 4.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the ratio of the amount of O-desmethylvenlafaxine or pharmaceutically acceptable salt thereof released at 24 hours in 900 mL 0.1 N HCl (pH 4.5) with 40% EtOH using USP Apparatus II at 50 rpm to the amount of O-desmethylvenlafaxine or pharmaceutically acceptable salt thereof released at 24 hours in 900 mL 0.1 N HCl (pH 4.5) using USP Apparatus II at 50 rpm is no more than 1.5:1; or no more than about 1.2:1.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 1 hour in 900 mL 0.1 N HCl (pH 4.5) with 40% EtOH using USP Apparatus II at 50 rpm is less than the amount of O-desmethylvenlafaxine or pharmaceutically acceptable salt thereof released at 1 hour in 900 mL 0.1 N HCl (pH 4.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 2 hours in 900 mL 0.1 N HCl (pH 4.5) with 40% EtOH using USP Apparatus II at 50 rpm is less than the amount of O-desmethylvenlafaxine or pharmaceutically acceptable salt thereof released at 2 hours in 900 mL 0.1 N HCl (pH 4.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 4 hours in 900 mL 0.1 N HCl (pH 4.5) with 40% EtOH using USP Apparatus II at 50 rpm is less than the amount of O-desmethylvenlafaxine or pharmaceutically acceptable salt thereof released at 4 hours in 900 mL 0.1 N HCl (pH 4.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 8 hours in 900 mL 0.1 N HCl (pH 4.5) with 40% EtOH using USP Apparatus II at 50 rpm is less than the amount of O-desmethylvenlafaxine or pharmaceutically acceptable salt thereof released at 8 hours in 900 mL 0.1 N HCl (pH 4.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 16 hours in 900 mL 0.1 N HCl (pH 4.5) with 40% EtOH using USP Apparatus II at 50 rpm is less than the amount of O-desmethylvenlafaxine or pharmaceutically acceptable salt thereof released at 16 hours in 900 mL 0.1 N HCl (pH 4.5) using USP Apparatus II at 50 rpm.

In certain embodiments, the present invention is directed to a controlled release oral dosage form comprising a therapeutically effective amount of O-desmethylvenlafaxine or a pharmaceutically acceptable salt thereof and a controlled release material; wherein the amount of O-desmethylvenlafaxine or pharmaceutically acceptable salt thereof released at 24 hours in 900 mL 0.1 N HCl (pH 4.5) with 40% EtOH using USP Apparatus II at 50 rpm is less than the amount of O-desmethylvenlafaxine or pharmaceutically acceptable salt thereof released at 24 hours in 900 mL 0.1 N HCl (pH 4.5) using USP Apparatus II at 50 rpm.

In certain embodiments, the present invention is directed to a controlled release oral dosage form as disclosed herein which provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter, of from about 10% to about 50% O-desmethylvenlafaxine or salt thereof released at 2 hours; from about 30% to about 65% O-desmethylvenlafaxine or salt thereof released at 4 hours; from about 40% to about 80% O-desmethylvenlafaxine or salt thereof released at 8 hours; and less than about 95% O-desmethylvenlafaxine or salt thereof released at about 16 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter of from about 10% to about 35% O-desmethylvenlafaxine or salt thereof released at 2 hours; from about 30% to about 55% O-desmethylvenlafaxine or salt thereof released at 4 hours; from about 50% to about 80% O-desmethylvenlafaxine or salt thereof released at 8 hours; and less than about 95% O-desmethylvenlafaxine or salt thereof released at about 16 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 thereafter of from about 10% to about 35% O-desmethylvenlafaxine or salt thereof released at 2 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter of from about 30% to about 55% O-desmethylvenlafaxine or salt thereof released at 4 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter of from about 40% to about 80% O-desmethylvenlafaxine or salt thereof released at 8 hours.

In certain embodiments of the present invention disclosed herein, the dissolution rate in-vitro of the dosage form, when measured by the USP Apparatus Type III at 37° C.±0.5 in 250 ml (per dissolution vessel) at 15 dpm, a pH of 1.5 for the first hour, with a switch to pH 4.5 for two hours, with a switch to pH 7.5 thereafter, is as follows: from 0% to about 50% O-desmethylvenlafaxine released at about 1 hour; from about 30% to about 60% at about 2 hours; from about 45% to about 80% released at about 4 hours; from about 60% to about 95% at about 8 hours; greater than about 50% released at about 16 hours; greater than about 80% O-desmethylvenlafaxine release at about 24 hours.

In certain embodiments of the present invention disclosed herein, the dissolution rate in-vitro of the dosage form, when measured by the USP Apparatus Type III at 37° C.±0.5 in 250 ml (per dissolution vessel) at 15 dpm, a pH of 1.5 for the first hour, with a switch to pH 4.5 for two hours, with a switch to pH 7.5 thereafter, is as follows: from about 15% to about 30% O-desmethylvenlafaxine released at about 1 hour; from about 20% to about 40% at about 2 hours; from about 30% to about 55% at about 4 hours; from about 50% to about 75% at about 8 hours; from about 80% to about 96% at about 16 hours; greater than about 80% O-desmethylvenlafaxine release at about 24 hours.

In certain embodiments of the present invention disclosed herein, the dissolution rate in-vitro of the dosage form, when measured by the USP Apparatus Type III at 37° C.±0.5 in 250 ml (per dissolution vessel) at 15 dpm, a pH of 1.5 for the first hour, with a switch to pH 4.5 for two hours, with a switch to pH 7.5 thereafter, is as follows: from about 20% to about 30% O-desmethylvenlafaxine released at about 1 hour; from about 30% to about 40% at about 2 hours; from about 40% to about 50% at about 4 hours; from about 60% to about 75% at about 8 hours; from about 80% to about 96% at about 16 hours; greater than about 80% O-desmethylvenlafaxine release at about 24 hours.

In certain embodiments, the dosage forms of the present invention are dose proportional or substantially dose proportional.

In certain embodiments, the dosage forms of the present invention are pseudo dose proportional.

In certain embodiments of the present invention, the gelling agent is included in an amount from about 40% to about 70%, and more preferably from about 50% to about 65%, by weight of the final product. The drug to gum ratio may be, e.g., from about 1:0.5 to about 1:6. More preferably, the drug to gum ratio is from about 1:2.1 to about 1:8.4.

In certain embodiments, the controlled release dosage form of the present invention comprises an effective amount of an ionizable gel strength enhancing agent to obtain a desirable increased gel strength due to cross-linking with the gelling agent.

In certain embodiments, the controlled release dosage form of the present invention further comprises a hydrophobic material in an amount effective to slow the hydration of the gelling agent when the formulation is exposed to fluids in an environment of use.

In certain preferred embodiments, the present invention is further directed to an extended release oral tablet comprising O-desmethylvenlafaxine or pharmaceutically acceptable salt thereof in an amount of from about 75 mg to about 375 mg dispersed in a matrix comprising (i) a gelling agent the gelling agent in an amount of from about 20% to about 70% by weight of the dosage form, (ii) an inert pharmaceutical diluent in an amount of from about 5% to about 40% by weight of the dosage form, and (iii) an ionizable gel strength enhancing agent in an amount of from about 4% to about 10% by weight of the dosage form; a hydrophobic coating coated over the matrix in an amount of from about 1% to about 20% by weight of the dosage form; wherein the formulation provides for the extended release of the O-desmethylvenlafaxine or pharmaceutically acceptable salt thereof and is suitable for once-a-day administration.

In certain embodiments, the dosage forms of the present invention comprise O-desmethylvenlafaxine in an amount of from about 25 mg to about 375 mg dispersed in a controlled release excipient comprising (i) locust bean gum in an amount of 10% to about 40% by weight of the oral dosage form and (ii) xanthan gum in an amount from about 10% to about 30% by weight of the oral dosage form, (iii) mannitol in an amount of from about 5% to about 40% by weight of the oral dosage form, and (iv) calcium sulfate dihydrate in an amount of about 4% to about 10% by weight of the oral dosage form.

In certain embodiments, the dosage form can comprise a hydrophobic material coated over the matrix in an amount, e.g., from about about 1% to about 25% in order to provide a further controlled release or to provide a delayed release with e.g., an enteric coating.

In certain embodiments, the controlled release dosage form of the present invention comprises from about 7% to about 35% O-desmethylvenlafaxine or salt thereof by weight of the dosage form or from about 15% to about 30% of the dosage form.

In certain embodiments, the controlled release dosage form of the present invention comprises from about 35% to about 85% gelling agent by weight of the dosage form or from about 50% to about 70% by weight of the dosage.

In certain embodiments, the controlled release dosage form of the present invention comprises from about 1% to about 25% hydrophobic material by weight of the dosage form, or from about 5% to about 15% hydrophobic material by weight of the dosage form.

The O-desmethylvenlafaxine formulations of the present invention which are resistant to alcohol induced dose dumping are not limited to dosage forms comprising a gelling agent. Any suitable controlled release technology which can provide the required resistance can be used. Such controlled release technology is described, e.g., in U.S. Patent Publication Nos. 2003/0118641; 2005/0163856; and 2004/0052731, the disclosures of which are hereby incorporated by reference.

In certain embodiments the invention is directed to a controlled release oral dosage form which dose proportional or substantially dose proportional.

In certain embodiments the invention is directed to a controlled release oral dosage form comprising O-desmethylvenlafaxine or a pharmaceutically acceptable salt thereof; wherein the dosage form is scored in order to divide the dosage form into equal or substantially equal divided doses.

In certain embodiments the invention is directed to a controlled release oral dosage form comprising a matrix comprising (i) O-desmethylvenlafaxine, or a pharmaceutically acceptable salt thereof and (ii) at least one controlled release excipient; the dosage form being scored in order to divide the dosage form into at least two substantially equal divided doses.

In certain embodiments the invention is directed to a method of titrating a patient in need of O-desmethylvenlafaxine therapy comprising: a) dividing a scored dosage form as disclosed herein into divided doses; b) administering a divided dose for at least one dosing interval to the patient; and c) increasing the dosage in a subsequent administration.

In certain embodiments the invention is directed to a method of titrating a patient in need of O-desmethylvenlafaxine therapy comprising: a) dividing a scored dosage form as disclosed herein into divided doses; and b) administering to a patient currently on O-desmethylvenlafaxine therapy a divided dose for at least one dosing interval in order to decrease the dosage to the patient.

In certain embodiments, the titration therapy disclosed herein results in decreased side effects associated with O-desmethylvenlafaxine therapy, e.g., nausea and/or vomiting as compared to immediate release therapy; as compared to initially administering the intended final dosage of O-desmethylvenlafaxine; or as compared to abrupt cessation of the O-desmethylvenlafaxine therapy.

By “controlled” it is meant for purposes of the present invention that the therapeutically active medicament is released from the formulation at a controlled rate.

The term “environmental fluid” is meant for purposes of the present invention to encompass, e.g., an aqueous solution, such as that used for in-vitro dissolution testing, or gastrointestinal fluid.

The term “mean” for purposes of the present invention, represents the arithmetic mean value measured across a set of values, e.g., across a patient population.

The term “dose proportional” for purposes of the present invention means that all active and inactive ingredients are in exactly the same proportion between different strengths (e.g., a tablet of 75-mg strength has all the inactive ingredients, exactly half that of a tablet of 150-mg strength, and twice that of a tablet of 37.5-mg strength).

The term “pseudo-dose proportional” means that either 1) the portion of the reduced active ingredient amount in the lower strength dosage form is replaced by an inert diluent such that the total tablet weight is same and the ratios of the inactive ingredients to total tablet weight except the inert diluent is the same or 2) the portion of the reduced active ingredient amount in the lower strength dosage form is not replaced by an inert diluent such that the total tablet weight is reduced equal to the lesser active ingredient and the ratios of the inactive ingredients to total tablet weight are the not the same.

### DETAILED DESCRIPTION

Venlafaxine has been shown to be a potent inhibitor of monoamine neurotransmitter uptake, a mechanism associated with clinical antidepressant activity. Due to its novel structure, venlafaxine has a mechanism of action unrelated to other available antidepressants, such as the tricyclic antidepressants desipramine, nortriptyline, protriptyline, imipramine, amitryptyline, trimipramine and doxepin.

It is believed that venlafaxine's mechanism of action is related to potent inhibition of the uptake of the monoamine neurotransmitters serotonin and norepinephrine. To a lesser degree, venlafaxine also inhibits dopamine reuptake, but it has no inhibitory activity on monoamine oxidase. O-desmethylvenlafaxine, venlafaxine's major metabolite in humans, exhibits a similar pharmacologic profile. Venlafaxine's ability to inhibit norepinephrine and serotonin (5-HT) uptake has been predicted to have an efficacy which rivals or surpases that of tricyclic antidepressants (Stuart A. Montgomery, M. D., J. Clin. Psychiatry, 54:3, March 1993).

Venlafaxine has been marketed as a once-a-day controlled release dosage form (i.e. Effexor XR) wherein the total daily dose is contained in one dosage form, with the intent that the agent is slowly released over the dosing interval. This dosage form, however, is susceptable to alcohol induced dose dumping, whereby the integrity of the controlled release mechanism is compromised in the presence of alcohol. This can result in an immediate release of the contents of the dosage form which was intended to release slowly over the entire dosing interval. This can result in “spiked” plasma levels of venlafaxine and may resut in increased side effects to the patient. The dosage forms of the present invention address this need in the art for a controlled release venlafaxine formulation which is resistant to alcohol induced dose dumping.

In certain other embodiments, the preferred active agent of the present invention is O-desmethylvenlafaxine in salt form such as O-desmethylvenlafaxine formate or O-desmethylvenlafaxine succinate in an amount that would be e.g., therapeutically equivalent to about 37.5 mg base, about 75 mg base or about 150 mg base.

In certain embodiments, the magnitude of a prophylactic or therapeutic dose of O-desmethylvenlafaxine or salt thereof, in the acute or chronic management of a disease will vary with the severity of the condition to be treated and the route of administration. The dose will also vary according to age, body weight, response, and the past medical history of the individual patient. In general the daily dose will lie within the range of from about 10 mg to about 1000 mg per day. Preferably, a daily dose range should be from about 50 mg to about 500 mg per day, more preferably, between about 75 mg and about 350 mg per day.

In certain embodiments where an O-desmethylvenlafaxine salt is utilzed, the daily dose will lie within the range of the salt form that is equivalent to from about 10 mg to about 1000 mg per day; preferably from about 50 mg to about 500 mg per day; and more preferably, between about 75 mg and about 350 mg per day.

In certain embodiments, the controlled release dosage form comprises O-desmethylvenlafaxine formate in an amount equivalent to from about 10 mg to about 1000 mg O-desmethylvenlafaxine base.

In certain preferred embodiments, the controlled release dosage form comprises O-desmethylvenlafaxine formate in an amount equivalent to from about 50 mg to about 500 mg O-desmethylvenlafaxine base.

In certain more preferred embodiments, the controlled release dosage form comprises O-desmethylvenlafaxine formate in an amount equivalent to from about 75 mg to about 375 mg O-desmethylvenlafaxine base.

In certain embodiments, the controlled release dosage form comprises O-desmethylvenlafaxine succinate in an amount equivalent to from about 10 mg to about 1000 mg O-desmethylvenlafaxine base.

In certain preferred embodiments, the controlled release dosage form comprises O-desmethylvenlafaxine succinate in an amount equivalent to from about 50 mg to about 500 mg O-desmethylvenlafaxine base.

In certain more preferred embodiments, the controlled release dosage form comprises O-desmethylvenlafaxine succinate in an amount equivalent to from about 75 mg to about 350 mg O-desmethylvenlafaxine base.

The O-desmethylvenlafaxine utilized in the invention can include the free base and any pharmaceutically acceptable salt forms of O-desmethylvenlafaxine, the racemate and its individual enantiomers, and O-desmethylvenlafaxine analogs, both as racemates and as their individual enantiomers. The use of O-desmethylvenlafaxine is also understood to include all crystalline and amorphous forms, and any polymorphs or hydrates.

The pharmaceutically acceptable salts of O-desmethylvenlafaxine include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, succinate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

O-desmethylvenlafaxine succinate is prepared as described in U.S. Pat. No. 6,673,838, which is incorporated by reference herein. The formate salt of O-desmethylvenlafaxine, described in published U.S. patent application Publn. No. U.S. 2003/0236309, which is incorporated by reference herein, can be prepared using similar techniques by substitution of the appropriate salt.

The polymorphs of O-desmethylvenlafaxine succinate are described in U.S. patent application Publn. No. US 20050096479. Crystalline forms of O-desmethylvenlafaxine formate are described in U.S. patent application Publn. No. 20030236309. All of these publications are incorporated by reference.

In certain embodiments of the present invention, the gelling agent comprises a polysaccharide, such as a heteropolysaccaride or a homopolysaccharide.

In a preferred embodiment, the gelling agent comprises a heteropolysaccharide and further comprises a homopolysaccharide gum capable of cross-linking the heteropolysaccharide gum when exposed to an environmental fluid. The preferred heteropolysaccharide gum is xanthan gum and the preferred homopolysaccharide gum is a galactomannan such as locust bean gum.

A non-limiting list of suitable hydrophobic materials which may be included in the dosage form of the present invention includes, such as gums, cellulose ethers, acrylic resins, protein derived materials, waxes, shellac, and oils such as hydrogenated castor oil and hydrogenated vegetable oil. Certain hydrophobic polymers include alkylcelluloses such as ethylcellulose, acrylic and methacrylic acid polymers and copolymers. Examples of acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Preferred waxes include for example natural and synthetic waxes, fatty acids, fatty alcohols, and mixtures of the same (e.g., beeswax, camauba wax, stearic acid and stearyl alcohol.

In certain preferred embodiments, the gelling agent of the present invention comprises a heteropolysaccharide such as xanthan gum, a homopolysaccharide such as locust bean gum, or a mixture of one or more hetero- and one or more homopolysaccharide(s). Heterodisperse excipients, previously disclosed in our U.S. Pat. Nos. 4,994,276, 5,128,143, and 5,135,757, may be utilized in the sustained release excipient of the present invention. For example, in certain embodiments of the present invention, the controlled release excipient comprises a gelling agent of both hetero- and homo- polysaccharides which exhibit synergism, e.g., the combination of two or more polysaccharide gums producing a higher viscosity and faster hydration than that which would be expected by either of the gums alone, the resultant gel being faster-forming and more rigid.

The term “heteropolysaccharide” as used in the present invention is defined as a water-soluble polysaccharide containing two or more kinds of sugar units, the heteropoly-saccharide having a branched or helical configuration, and having excellent water-wicking properties and immense thickening properties.

An especially preferred heteropolysaccharide is xanthan gum, which is a high molecular weight (>10⁶) heteropolysaccharide. Other preferred heteropolysaccharides include derivatives of xanthan gum, such as deacylated xanthan gum, the carboxymethyl ether, and the propylene glycol ester.

The homopolysaccharide materials used in the present invention that are capable of cross-linking with the heteropolysaccharide include the galactomannans, i.e., polysaccharides that are composed solely of mannose and galactose. A possible mechanism for the interaction between the galactomannan and the heteropolysaccharide involves the interaction between the helical regions of the heteropolysaccharide and the unsubstituted mannose regions of the galactomannan. Galactomannans that have higher proportions of unsubstituted mannose regions have been found to achieve more interaction with the heteropolysaccharide. Hence, locust bean gum, which has a higher ratio of mannose to galactose, is especially preferred.

The combination of xanthan gum with locust bean gum is an especially preferred gum combination for use in the sustained release excipient of the present invention.

In certain preferred embodiments of the present invention, the controlled release properties of the final product are optimized when the ratio of heteropolysaccharide gum to homopolysaccharide gum is from about 1:3 to about 3:1. However, the may comprise from about 1% to about 99% by weight heteropolysaccharide gum and from about 99% to about 1% by weight homopolysaccharide gum.

The combination of any homopolysaccharide gums know to produce a synergistic effect when exposed to aqueous solutions may be used in accordance with the present invention. It is also possible that the type of synergism which is present with regard to the gum combination of the present invention could also occur between two homogeneous or two heteropolysaccharides. Other acceptable gelling agents which may be used in the present invention include those gelling agents well-known in the art. Examples include vegetable gums such as alginates, carrageenan, pectin, guar gum, modified starch, hydroxypropylmethylcellulose, methylcellulose, and other cellulosic materials such as and hydroxypropyl cellulose. This list is not meant to be exclusive.

The dosage form of the present invention can further comprise an inert diluent. The inert diluent of the sustained release excipient preferably comprises a pharmaceutically acceptable saccharide, including a monosaccharide, a disaccharide, or a polyhydric alcohol, and/or mixtures of any of the foregoing. Examples of suitable inert pharmaceutical diluents include sucrose, dextrose, lactose, mannitol, microcrystalline cellulose, fructose, xylitol, sorbitol, starches, mixtures thereof and the like. However, it is preferred that a soluble pharmaceutical filler such as lactose, dextrose, mannitol, sucrose, or mixtures thereof be used. In certain especially preferred embodiments the diluent or filler is mannitol.

In certain embodiments, it is possible to dry mix the ingredients of the dosage form without utilizing a wet granulation step. This procedure may be utilized, for example, where a wet granulation is to be accomplished when the active ingredient is directly added to the ingredients of the sustained release excipient. On the other hand, this procedure may also be used where no wet granulation step whatsoever is contemplated. If the mixture is to be manufactured without a wet granulation step, and the final mixture is to be tableted, it is preferred that all or part of the inert diluent comprise a pre-manufactured direct compression diluent. Such direct compression diluents are widely used in the pharmaceutical arts, and may be obtained from a wide variety of commercial sources. Examples of such pre-manufactured direct compression excipients include Emcocel® (microcrystalline cellulose, N.F.), Emdex® (dextrates, N.F.), and Tab-Fine® (a number of direct-compression sugars including sucrose, fructose and dextrose), all of which are commercially available from JRS Pharma LP, Patterson, N.Y.). Other direct compression diluents include Anhydrous lactose (Lactose N.F., anhydrous direct tableting) from Sheffield Chemical, Union, N.J. 07083; Elcems® G-250 (powdered cellulose), N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltodextrin) from Grain Processing Corp., Muscatine, Iowa 52761; Neosorb 600 (Sorbitol, N.F., direct-compression from Roquet Corp., 645 5th Ave., New York, N.Y. 10022; Nu-Tab® (Compressible sugar, N.F.) from Ingredient Technology, Inc., Pennsauken, N.J. 08110; Polyplasdone XL® (Crospovidone, N.F., cross-linked polyvinylpyrrolidone) from ISP Corp., Wayne, N.J. 07470; Primojele (Sodium starch glycolate, N.F., carboxymethyl starch) from Generichem Corp., Little Falls, N.J. 07424; Solka Floc® (Cellulose floc); Spray-dried lactose® (Lactose N.F., spray dried) from Foremost Whey Products, Baraboo, Wis. 53913 and DMV Corp., Vehgel, Holland; and Sta-Rx 1500® (Starch 1500) (Pregelatinized starch, N.F., compressible) from Colorcon, Inc., West Point, Pa. 19486.

In further embodiments of the present invention, the directly compressible inert diluent which is used in conjunction with the sustained release excipient of the present invention is an augmented microcrystalline cellulose as disclosed in U.S. patent application Ser. No. 08/370,576, filed Jan. 9, 1995, and entitled “PHARMACEUTICAL EXCIPIENT HAVING IMPROVED COMPRESSIBILITY”, by J. Staniforth, B. Sherwood and E. Hunter, hereby incorporated by reference in its entirety. The augmented microcrystalline cellulose described therein is commercially available under the tradename “Prosolv” from JRS Pharma LP.

The controlled release excipients prepared in accordance with the present invention may be prepared according to any agglomeration technique to yield an acceptable sustained release excipient product. For example, in wet granulation techniques, the desired amounts of the heteropolysaccharide gum, the homopolysaccharide gum, and the inert diluent are mixed together and thereafter a moistening agent such as water, propylene glycol, glycerol, alcohol or the like is added to prepare a moistened mass. Next, the moistened mass is dried. The dried mass is then milled with conventional equipment into granules. Thereafter, the excipient product is ready to use. The granulate form has certain advantages including the fact that it can be optimized for flow and compressibility; it can be tableted, formulated in a capsule, extruded and spheronized with an active medicament to form pellets, etc.

In certain embodiments of the invention where the controlled release excipient comprises a heteropolysaccharide, a homopolysaccharide, or both, a release-modifying agent may also be incorporated in the formulations (e.g., in the sustained release excipient) of the present invention. Such release-modifying agents and pre-manufactured excipients disclosed in our U.S. Pat. Nos. 5,455,046; 5,512,297; 5,554,387; 5,667,801; 5,846,563; 5,773,025; 6,048,548; 5,662,933; 5,958,456; 5,472,711; 5,670,168; and 6,039,980 may be utilized in the present invention.

Thus, for example, the release-modifying agent may comprise an ionizable gel-strength enhancing agent. The ionizable gel strength-enhancing agent that is optionally used in conjunction with the present invention may be monovalent or multivalent metal cations. The preferred salts are the inorganic salts, including various alkali metal and/or alkaline earth metal sulfates, chlorides, borates, bromides, citrates, acetates, lactates, etc. Specific examples of suitable ionizable gel strength enhancing agent include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof. Multivalent metal cations may also be utilized. However, the preferred ionizable gel strength-enhancing agents are bivalent. Particularly preferred salts are calcium sulfate and sodium chloride. In a particular preferred embodiment, the ionizable gel strength-enhancing agent is calcium sulfate dihydrate. The ionizable gel strength enhancing agents of the present invention are added in an amount effective to obtain a desirable increased gel strength due to the cross-linking of the gelling agent (e.g., the heteropolysaccharide and homopolysaccharide gums). In certain embodiments, the ionizable gel strength-enhancing agent is included in the controlled release excipient of the present invention in an amount from about 1 to about 20% by weight of the controlled release excipient, and in an amount 0.5% to about 16% by weight of the final dosage form.

In certain embodiments, the dosage form of the present invention can comprise a surfactant. Surfactants that may be used in the present invention generally include pharmaceutically acceptable anionic surfactants, cationic surfactants, amphoteric (amphipathic/ amphophilic) surfactants, and non-ionic surfactants. Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates (including sodium lauryl sulfate (SLS)), ethoxylated alkyl sulfates, ester linked sulfonates (including docusate sodium or dioctyl sodium succinate (DSS)), alpha olefin sulfonates, and phosphated ethoxylated alcohols.

Suitable pharmaceutically acceptable cationic surfactants include, for example, monoalkyl quaternary ammonium salts, dialkyl quaternary ammonium compounds, amidoamines, and aminimides.

Suitable pharmaceutically acceptable amphoteric (amphipathic/amphophilic) surfactants, include, for example, N-substituted alkyl amides, N-alkyl betaines, sulfobetaines, and N-alkyl β-aminoproprionates.

Other suitable surfactants for use in conjunction with the present invention include polyethyleneglycols as esters or ethers. Examples include polyethoxylated castor oil, polyethoxylated hydrogenated castor oil, or polyethoxylated fatty acid from castor oil or polyethoxylated fatty acid from hydrogenated castor oil. Commercially available surfactants that can be used are known under trade names Cremophor, Myrj, Polyoxyl 40 stearate, Emerest 2675, Lipal 395 and PEG 3350.

Other release-modifying pharmaceutically acceptable agents that may be added in appropriate quantities for their particular ability to modify dissolution rates include, for example: stearic acid, metallic stearates, stearyl alcohol, hydrogenated cotton seed oil, sodium chloride and certain disintegrants.

The quantity of such release-modifying agent employed depends on the release characteristics required and the nature of the agent. For the sustained release formulation according to the invention, the level of release-modifying agents used may be from about 0.1 to about 25%, preferably from about 0.5 to about 20% by weight of the total composition.

In certain embodiments of the present invention a hydrophobic material is added to the formulation. This may be accomplished by granulating the sustained release excipient with a solution or dispersion of hydrophobic material prior to the incorporation of the medicament. The hydrophobic material may be selected from ethylcellulose, acrylic and/or methacrylic acid polymers or copolymers, hydrogenated vegetable oils, zein, as well as other pharmaceutically acceptable hydrophobic materials known to those skilled in the art. Other hydrophobic cellulosic materials such as other alkyl celluloses may also be used. The amount of hydrophobic material incorporated into the sustained release excipient is that which is effective to slow the hydration of the gums without disrupting the hydrophilic matrix formed upon exposure to an environmental fluid. In certain preferred embodiments of the present invention, the hydrophobic material may be included in the sustained release excipient in an amount from about 1% to about 20% by weight of the sustained release excipient. More preferably, the hydrophobic material may be included in the sustained release excipient in an amount from about 1% to about 10%, and most preferably from about 1% to about 5%, by weight of the final formulation. The hydrophobic material may be dissolved in an organic solvent or dispersed in an aqueous solution for incorporation into the formulation.

Preferably, the controlled release excipients of the invention have uniform packing characteristics over a range of different particle size distributions and are capable of processing into tablets using either direct compression, following addition of drug and lubricant powder, conventional wet granulation, or spray granulation techniques.

In certain embodiments, the oral dosage form of the present invention may be prepared as granules, spheroids, matrix multiparticulates, etc. which comprise venlafaxine or salt thereof in a sustained release matrix, which may be compressed into a tablet or encapsulated.

In certain embodiments, the complete mixture is in an amount sufficient to make a uniform batch of tablets and is subjected to tableting in a conventional production scale tableting machine at normal compression pressure, e.g., about 2000-1600 lbs/sq in. The average tablet weight may be from about 100 mg to 1500 mg.

In certain embodiments of the present invention, the granules, spheroids, matrix multiparticulates, or tableted formulation are coated with a coating layer which may be comprised of a polymer, mixture of polymers, synthetic and/or naturally occurring, that are freely permeable, slightly permeable, water soluble, water insoluble, and polymers whose permeability and/or solubility is affected by pH.

The coating can comprise a hydrophobic material such as those described above. For example, the hydrophobic material may be a hydrophobic polymer, acrylic and/or methacrylic acid polymers or copolymers, hydrogenated vegetable oils, zein, mixtures thereof, as well as other pharmaceutically acceptable hydrophobic materials known to those skilled in the art. Hydrophobic cellulosic materials such as alkyl celluloses may also be used. In certain embodiments the hydrophobic material in the coating is in an amount of from about 2% to about 15% by weight of the final formulation, preferably from about 2% to about 10% by weight of the final formulation. An especially preferred hydrophobic material is ethylcellulose. Ethylcellulose is commercially available as Aquacoat® (aqueous dispersion of ethylcellulose available from FMC) and Surelease® (aqueous dispersion of ethylcellulose available form Colorcon). In certain preferred embodiments, the ethylcellulose (e.g., aqueous dispersion of ethylcellulose) is mixed with a hydrophilic coating material such a hydroxypropylmethylcellulose (commercially available as Opadry® commercially available from Colorcon, West Point, Pa.) prior to coating the final dosage form.

In other embodiments of the present invention, the hydrophobic material is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and mixtures thereof.

In certain embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit(® from Rohm Pharma.

In certain embodiments, a combination of any of the aforementioned hydrophobic materials may be used.

In embodiments of the present invention where the coating comprises an aqueous dispersion of a hydrophobic material, the inclusion of an effective amount of a plasticizer in the aqueous dispersion of hydrophobic material will further improve the physical properties of the sustained release coating.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is also a preferred plasticizer for the acrylic polymers of the present invention.

Such suitable polymers for inclusion into the coating layer preferably slow the release profile of the dosage form.

In other embodiments of the present invention, the coating layer may comprise an enteric coating material in addition to or instead of the hydrophobic polymer coating. Examples of suitable enteric polymers include cellulose acetate phthalate, hydroxy-propylmethylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit™ L30D55.

In further embodiments, the dosage form may be coated with a hydrophilic coating in addition to or instead of the above-mentioned coatings. An example of a suitable material which may be used for such a hydrophilic coating is hydroxypropylmethylcellulose (e.g., Opadry® as described above).

The coating layer may be applied in any pharmaceutically acceptable manner known to those skilled in the art. For example, in one embodiment, the coating is applied via a fluidized bed or in a coating pan. For example, the coated tablets may be dried, e.g., at about 60-70° C. for about 3-4 hours in a coating pan. The solvent for the hydrophobic polymer or enteric coating may be organic, aqueous, or a mixture of an organic and an aqueous solvent. The organic solvents may be, e.g., isopropyl alcohol, ethanol, and the like, with or without water.

In additional embodiments of the present invention, a support platform is applied to the tablets manufactured in accordance with the present invention. Suitable support platforms are well known to those skilled in the art. An example of suitable support plat-form is set forth, e.g., in U.S. Pat. No. 4,839,177, hereby incorporated by reference. In that patent, the support platform partially coats the tablet, and consists of a polymeric material insoluble in aqueous liquids. The support platform may, for example, be designed to maintain its impermeability characteristics during the transfer of the therapeutically active medicament. The support platform may be applied to the tablets, e.g., via compression coating onto part of the tablet surface, by spray coating the polymeric materials comprising the support platform onto all or part of the tablet surface, or by immersing the tablets in a solution of the polymeric materials.

The support platform may have a thickness of, e.g., about 2 mm if applied by compression, and about 10 μm if applied via spray-coating or immersion-coating.

Generally, in embodiments of the invention wherein a coating comprising a hydrophobic material or enteric coating material is applied to a core, the cores are coated to a weight gain from about 1 to about 20%, and in certain embodiments preferably from about 5% to about 15%, in certain preferred embodiments from about 7% to about 15%, and in a particular preferred embodiment, about 11%. In certain embodiments, the coating comprising the hydrophobic material is in an amount of from about 1% to about 20, preferably from about 2% to about 15% by weight of the final formulation.

Additionally, the cores may optionally be coated with a color coat that rapidly disintegrates or dissolves in water or the environment of use. The color coat may be a conventional sugar or polymeric film coating which is applied in a coating pan or by conventional spraying techniques. Preferred materials for the color coat are commercially available under the Opadry tradename (e.g, Opadry® II White, Opadry® II Blue). The color coat may be applied directly onto the tablet core, or may be applied after a coating as described above. Generally, the color coat surrounding the core will comprise from about 1 to 5% preferably about 2 to 4% based on the total weight of the tablet.

An effective amount of any generally accepted pharmaceutical lubricant or mixture of lubricants, including the calcium or magnesium soaps may be added to the above-mentioned ingredients of the formulation at the time the medicament is added, or in any event prior to compression into a solid dosage form. Preferably the lubricant is in an amount of from about 0.5% to about 10%, more preferably from about 0.5% to about 5% by weight of the final formulation. An example of a suitable lubricant is magnesium stearate in an amount of about 0.5% to about 3% by weight of the solid dosage form. An especially preferred lubricant is sodium stearyl fumarate, NF, commercially available under the trade name Pruv® from JRS Pharma LP. Another preferred lubricant is talc.

An effective amount of any generally acceptable pharmaceutical glidant or mixture of glidants may also be added to the formulation at the time the medicament is added, or in any event prior to compression into a solid dosage form, including colloidal silicon dioxide, talc, silicon dioxide, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch, starch 1500, magnesium lauryl sulfate, and magnesium oxide. In certain embodiments, a glidant may also be added to the material to be coated prior to application. Preferably the glidant is in an amount of from about 0.5% to about 10%, preferably from about 2% to about 8% by weight of the final formulation.

In certain embodiments, defoaming agents, also known as antifoaming agents, are included in the dosage forms of the present invention. The antifoaming agents are substances used to reduce foaming due to mechanical agitation or to gases, nitrogenous materials or other substances which may interfere during processing. Examples include metallic salts such as sodium chloride; C₆ to C₁₂ alcohols such as octanol; sulfonated oils; silicone ethers such as simethicone; organic phosphates and the like. The amount of antifoaming agent in the composition can range from about 0.005 to about 5%, preferably from about 0.01 to about 2%.

In certain embodiments utilizing an inert diluent, additional inert diluent may also be incorporated in the sustained release oral dosage form when mixing the sustained release excipient with the venlafaxine hydrochloride. The inert diluent may be the same or different inert diluent that is incorporated into the sustained release excipient. Other pharmaceutically acceptable diluents and excipients that may be used to formulate oral dosage forms of the present invention are described in the Handbook of Pharmaceutical Excipients, American Pharmaceutical Association (1986).

In certain embodiments, the controlled release excipients of the present invention are prepared via a wet granulation method. However, the controlled release excipients prepared in accordance with the present invention may be prepared according to any agglomeration technique to yield an acceptable excipient product. For example, in wet granulation techniques utilizing heterpolysaccharide and homopolysaccharide gums, the desired amounts of the gums, optional cationic cross-linking agent and the inert diluent are mixed together and thereafter a moistening agent such as water, propylene glycol, glycerol, alcohol or the like is added to prepare a moistened mass. Next, the moistened mass is dried. The dried mass is then milled with conventional equipment to obtain the desired particle size.

Once the sustained release excipient of the present invention has been prepared, it is then possible to blend the same with the venlafaxine or salt thereof, e.g., in a V-blender and compress the blend into an extended release oral tablet.

In certain preferred embodiments, the mixture of sustained release excipient and the active ingredient e.g., venlafaxine hydrochloride (and optionally additional diluent and excipients) may be spray granulated with a solution or suspension of e.g., a cellulose derivative such as an alkylcellulose, hydroxyalkylcellulose, hydroxyalkylalkylcellulose, or mixtures thereof. Preferably, the cellulose derivative is an alkylcellulose such as ethylcellulose, methylcellulose, and the like; a hydroxylalkylcellulose such as hydroxyethylcellulose, hydroxypropylcellulose, and the like; a hydroxylalkylalkylcellulose such as hydroxypropylmethylcellulose, hydroxyethylmethylcellulose, and the like; or mixtures thereof. In certain alternate embodiments, the sustained release excipient may be spray granulated with the cellulose derivative prior to incorporation of the active ingredient, e.g., venlafaxine hydrochloride. Preferably the cellulose derivative used in the spray granulation (e.g., hydroxypropylmethylcellulose) is in the final formulation in an amount of from about 1% to about 10%, preferably from about 2 to about 6% by weight of the final formulation. Preferably the inclusion of the cellulose derivative via spray granulation aids the processing (e.g., tableting) of the formulations (e.g., decreases sticking of granulated powders to the tablet press).

Preferably the granules are compressed into tablets. Although tablets are preferred dosage forms of the present invention, the ingredients may also be formulated in a capsule, extruded and spheronized with an active medicament to form pellets, etc.

In certain preferred embodiments, after the granules are compressed into tablets, the tablets are overcoated with one or more of the coatings described above. In certain embodiments, however, the matrix is capable of providing the desired controlled release without a controlled or delayed release coating.

In tablet formulations, the tablets have a hardness from about 7 to about 20 kP.

### DETAILED DESCRIPTION OF PREFERRED EMBODIMENTS

The following examples illustrate various aspects of the present invention. They are not to be construed to limit the claims in any manner whatsoever.

### EXAMPLES 1-7 (Prophetic)

In Examples 1-7, a sustained release excipient (70% gum) in accordance with the present invention is prepared. The sustained release excipient is prepared by dry blending the requisite amounts of xanthan gum, locust bean gum, calcium sulfate and mannitol in a high speed mixer/granulator. While running choppers/impellers, water is added to the dry blended mixture, and granulated. The granulation is then dried in a fluid bed dryer to a LOD (loss on drying) of less than about 10% by weight (e.g., 4-7% LOD). The granulation is then milled using comminuting machine. The ingredients of the sustained release excipient of Examples 1-7 are listed in Table 1 below:

TABLE 1

| Component | Amount (%) |
|---|---|
| Xanthan Gum | 28% |
| Locust Bean Gum | 42% |
| Calcium Sulfate Dihydrate | 10% |
| Mannitol, USP | 20% |
| Water* | q.s. |

*Removed during processing

The formulations of Examples 1-7 are set forth below in Table 2:

TABLE 2

| | Example 1 | Example 2 | Example 3 | Example 4 | Example 5 | Example 6 | Example7 |
|---|---|---|---|---|---|---|---|
| Ingredient | mg/tab | mg/tab | mg/tab | mg/tab | mg/tab | mg/tab | mg/tab |
| Sustained | 638.2 | 450 | 450 | 450 | 450 | 112.5 | 225 |
| Release | (79.8%) | (73.8%) | (74.6%) | (85.3%) | (91.8%) | (73.8%) | (73.8%) |
| Excipient (70%) | 150 | 150 | 150 | 75 | 37.5 | 37.5 | 75 |
| O- | (18.8%) | (24.6%) | (24.9%) | (14.2%) | (7.7%) | (24.6%) | (24.6%) |
| desmethylvenlafaxine | | | | | | | |
| formate | | | | | | | |
| Sodium stearyl | 11.8 | 10 | 3 | 2.6 | 2.5 | 2.5 | 5 |
| fumarate | (1.5%) | (1.6%) | (0.5%) | (0.5%) | (0.5%) | (1.6%) | (1.6%) |
| Total Weight | 800 | 610 | 603 | 527.6 | 490 | 152.5 | 305 |
| Active:Gum | 1:2.98 | 1:2.1 | 1:2.1 | 1:4.2 | 1:8.4 | 1:2.1 | 1:2.1 |
| Tooling Size | 0.280″ × | 0.2500″ × | 0.2500″ × 0.7250″ | 0.2500″ × 0.6750″ | 0.2500″ × 0.6750″ | ♯9/32♭″ | ♯5/16♭″ |
| | 0.750″ | 0.7250″ | | | | | |

NOTE:
Range of Active:Gum used in Examples 1-7 is 1:2.1 to 1:8.4.

The formulations of Examples 1-7 are prepared as follows:

1. O-desmethylvenlafaxine formate is sifted through a sieve and mixed with the sustained release excipient in a high shear granulator.

2. The dry blend of step 1 is granulated with water until consistent granulation is achieved.

3. The wet mass of step 2 is dried in fluidized bed dryer.

4. The dried granules of step 3 is passed through a Fitzmill.

5. Sodium stearyl fumarate is passed through a sieve and mixed with the milled granules of step 4.

6. The lubricated blend of step 5 is compressed to make the tablets of about 800 mg for Example 1 and 610 mg for Example 2.

### EXAMPLES 8 to 14 (Prophetic)

Examples 8-14 are prepared in accordance with Examples 1-7 above using O-desmethylvenlafaxine succinate rather than O-desmethylvenlafaxine formate.

Examples 1-14 are expected to exhibit reduced alcohol induced dose dumping in accordance with the present invention.

1. A controlled release oral dosage form comprising:
a therapeutically effective amount of O-desmethylvenlafaxine or a pharmaceutically acceptable salt thereof and a controlled release material;
wherein the amount of O-desmethylvenlafaxine or pharmaceutically acceptable salt thereof released at 1 hour in 900 mL 0.1 N HCl (pH 1.5) with 40% EtOH using USP Apparatus II at 50 rpm is within 25% of the amount of O-desmethylvenlafaxine or pharmaceutically acceptable salt thereof released at 1 hour in 900 mL 0.1 N HCl (pH 1.5) using USP Apparatus II at 50 rpm.

2. The controlled release dosage form of claim 1, wherein the amount of O-desmethylvenlafaxine or pharmaceutically acceptable salt thereof released at 4 hours in 900 mL 0.1 N HCl (pH 1.5) with 40% EtOH using USP Apparatus II at 50 rpm is within 25% of the amount of O-desmethylvenlafaxine or pharmaceutically acceptable salt thereof released at 4 hours in 900 mL 0.1 N HCl (pH 1.5) using USP Apparatus II at 50 rpm.

3-7. (canceled)

8. The controlled release dosage form of claim 1 comprising a matrix comprising the O-desmethylvenlafaxine or pharmaceutically acceptable salt thereof dispersed in the controlled release material.

9. The controlled release dosage form of claim 8, wherein the controlled release material is a gelling agent.

10. The controlled release dosage form of claim 9, wherein said gelling agent comprises a polysaccharide.

11. The controlled release dosage form of claim 10, wherein said polysaccharide is a heteropolysaccharide gum.

12. The controlled release dosage form of claim 1l, wherein said gelling agent further comprises a homopolysaccharide gum capable of cross-linking the heteropolysaccharide gum when exposed to an environmental fluid.

13. The controlled release oral dosage form of claim 12, wherein said heteropolysaccharide gum is xanthan gum.

14. The controlled release oral dosage form of claim 12, wherein said homopolysaccharide gum is locust bean gum.

15. The controlled release oral dosage form of claim 9, wherein said matrix further comprises a hydrophobic material.

16. The controlled release oral dosage form of claim 15, wherein said hydrophobic material is selected from the group consisting of a hydrophobic polymer, a cellulosic material, an acrylic polymer, a methacrylic acid polymer, a methacrylic copolymer, hydrogenated vegetable oils, zein, and mixtures thereof.

17. The controlled release oral dosage form of claim 16, wherein said hydrophobic material comprises ethylcellulose.

18. The controlled release oral dosage form claim 9,,further comprising an ionizable gel strength enhancing agent capable of crosslinking with said gelling agent and increasing the gel strength when the dosage form is exposed to an environmental fluid.

19. The controlled release oral dosage form of claim 1 , wherein said ionizable gel strength enhancing agent comprises an alkali metal or an alkaline earth metal sulfate, chloride, borate, bromide, citrate, acetate, or lactate.

20. The controlled release oral dosage form of claim 18, wherein said ionizable gel strength enhancing agent is selected from the group consisting of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof.

21. The controlled release oral dosage form of claim 18., wherein said ionizable gel strength enhancing agent comprises calcium sulfate.

22. The controlled release dosage form of claim 9, wherein said matrix further comprises an inert pharmaceutical diluent.

23. The controlled release dosage form of claim 22, wherein said inert diluent is selected from the group consisting of a monosaccharide, a disaccharide, a polyhydric alcohol, and mixtures thereof.

24. The controlled release dosage form of claim 23, wherein said inert diluent comprises mannitol.

25. The controlled release dosage form of claim 22, wherein the ratio of said inert diluent to said gelling agent is from about 1:5 to about 5:1.

26. The controlled release dosage form of any of claim 23, wherein said matrix further comprises from about 1 to about 20% by weight microcrystalline cellulose.

27. The controlled release dosage form of any of claim 23, wherein said matrix further comprises from about 1 to about 20% by weight silicified microcrystalline cellulose.

28-30. (canceled)

31. The controlled release dosage form of claim 1 comprising O-desmethylvenlafaxine formate.

32. The controlled release dosage form of claim 1 comprising O-desmethylvenlafaxine succinate.

33-51. (canceled)

52. A controlled release oral dosage form comprising:
a therapeutically effective amount of O-desmethylvenlafaxine or a pharmaceutically acceptable salt thereof and a controlled release material;
wherein the amount of O-desmethylvenlafaxine or pharmaceutically acceptable salt thereof released at 1 hour in 900 mL 0.1 N HCl (pH 1.5) with 40% EtOH using USP Apparatus II at 50 rpm is less than the amount of O-desmethylvenlafaxine or pharmaceutically acceptable salt thereof released at 1 hour in 900 mL 0.1 N HCl (pH 1.5) using USP Apparatus II at 50 rpm.

53-65. (canceled)
